(19) United States
(12) Patent Application Publication
(10) Pub. No.: US 2006/0233723 A1
(43) Pub. Date: Oct. 19, 2006

(54) Mouth and/or throat wash

(76) Inventor: Idalecio Bossa Fernandes Maleno, East Sussex (GB)

Correspondence Address: BLANK ROME LLP, 600 NEW HAMPSHIRE AVENUE, N.W., WASHINGTON, DC, 20037, US

(21) Appl. No.: 10/562,320

(22) PCT Filed: Jul. 1, 2004
(86) PCT No.: PCT/GB04/02873

§ 371 Date: Apr. 13, 2006

(30) Foreign Application Priority Data
Jul. 1, 2003 (GB) 0315269.1

(51) Int. Cl. A61K 8/19

(52) U.S. Cl. 424/49

(57) ABSTRACT

A mouth and/or throat wash which comprises an aqueous solution produced by immersing a permeable sac containing a measured quality of a mix of potassium nitrate, activated charcoal and particulate sulphur in a measured quantity of boiling water. The sac is removed from the boiling water after a predetermined period of time, and the solution is allowed to cool.

This invention relates to a mouth and/or throat wash for treating inter alia infections of the mouth or throat.

In one aspect, the invention provides a mouth and/or throat wash which comprises an aqueous solution produced by immersing a permeable sac containing a measured quantity of a mix of potassium nitrate, activated charcoal and particulate sulphur in a measured quantity of boiling water, removing the sac from the boiling water after a predetermined period of time, and allowing the solution to cool.

In another aspect the invention provides a water immersible permeable sealed sac containing a measured mix of potassium nitrate, activated charcoal and particulate sulphur, the sac being usable in a treatment for infections of the mouth or throat.

In a further aspect, the invention provides a mouth and/or throat wash which comprises an aqueous solution produced by immersing a permeable sealed sac containing measured quantities of at least two of potassium nitrate, activated charcoal and particulate sulphur in a measured quantity of boiling water for a predetermined period of time, discarding the sac and allowing the solution to cool.

In a still further aspect, the invention- provides a method of producing a mouth or throat wash which comprises placing a permeable sealed sac containing a measured quantity of a mix of potassium nitrate, activated charcoal and particulate sulphur in a measured quantity of water, bringing the water to the boil, removing the sac from the boiling water after a given period of time, and allowing the water containing a solution of the mix to cool.

In a still further embodiment, the invention provides a mouth and/or throat wash which comprises placing a permeable sealed sac containing a measured quantity of a mix of at least two of potassium nitrate, activated charcoal and particulate sulphur in a measured quantity of water, bringing the water to the boil, removing the sac from the boiling water after a given period of time, and allowing the water now containing a solution of the contents of the sac to cool.

By “sac” is meant a sealed bag, sachet or like container.

Typically, compounds in accordance with the invention contain between 70 and 80% by weight potassium nitrate, 10 to 25% activated charcoal and between 5 and 20% particulate sulphur.

In one embodiment, the proportions of potassium nitrate, activated charcoal and particulate sulphur are 75:15:10.

Typically a permeable sac contains between 5 and 200 g of the mix. Preferred ranges are between 30 and 150 g and between 55 and 10 g of the mix. The quantity of water in which the sac is immersed is typically between 50 to 750 ml; preferred quantities are between 250 and 500 ml and 300 and 420 ml.

The predetermined period of time may be between 2 and 5 minutes. Preferably, the time period is between 3 and 4 minutes.

The sac may comprise a woven cloth of, for example, linen or like material. Other sac materials may however be used, these including paper.

In one example of a mouth or throat wash in accordance with the invention, approximately 60 g of a mix of potassium nitrate, activated charcoal and particulate sulphur was placed in a permeable cloth sac. The proportions by weight of potassium nitrate, activated charcoal and particulate sulphur in the mix were 75:15:10. The open end of the sac was sealed and the sac was placed In approximately 350 ml of cold water. The water was brought to the boil and the sac was left in the boiling water for approximately 4 minutes. During the boiling process, a solution of the mix diffused through the pores of the sac to create within the water a weak solution. The sac was then removed and discarded. When cooled to an acceptable temperature, the water was used as a gargle for treating a patient having a throat infection. The gargle was used when warm; preferably, the temperature of the mouthwash when taken should be as hot as is comfortable for the user. After taking 5 gargles over a time period of 15 to 30 minutes, the patient's throat infection was relieved.

In another example, a permeable cloth sac was filled with approximately 75 g of potassium nitrate, activated charcoal and particulate sulphur in respective proportions by weight of 75:15:10 and the sac sealed. The sac was placed in 300 ml of cold water which was brought to the boil. After 3 minutes the sac was removed from the boiling water and discarded. After cooling to an acceptable warm temperature, the water was used effectively as a mouth wash to relieve the infection after 4 gargles over a time period of around 5 minutes.

If necessary, a flavouring may be added to the mouthwash.

It will be appreciated that the foregoing is merely exemplary of medicinal compounds In accordance with the invention and that modifications can readily be made thereto without departing from the true scope of the invention.

1. A mouth and/or throat wash which comprises an aqueous solution produced by immersing a permeable sac containing a measured quantity of a mix of potassium nitrate, activated charcoal and particulate sulphur in a measured quantity of boiling water, removing the sac from the boiling water after a predetermined period of time, and allowing the solution to cool.

2. A water immersible permeable seated sac containing a measured mix of potassium nitrate, activated charcoal and particulate sulphur, the sac being usable in a treatment for infections of the mouth or throat.

3. A mouth and/or throat wash which comprises an aqueous solution produced by immersing a permeable sealed sac containing a mix of measured quantities of at least two of potassium nitrate, activated charcoal and particulate sulphur in a measured quantity of boiling water for a predetermined period of time, discarding the sac and allowing the solution to cool.

4. A method of producing a mouth or throat wash which comprises placing a permeable sealed sac containing a measured quantity of a mix of potassium nitrate, activated charcoal and particulate sulphur in a measured quantity of water, bringing the water to the boil, removing the sac from the boiling water after a given period of time, and allowing the water containing a solution of the mix to cool.

5. A mouth and/or throat wash which comprises placing a permeable sealed sac containing a measured quantity of a mix of at least two of potassium nitrate, activated charcoal and particulate sulphur in a measured quantity of water, bringing the water to the boil, removing the sac from the boiling water after a given period of time, and allowing the water now containing a solution of the contents of the sac to cool.

6. A mouth wash, sealed sac or method as claimed in claim 1 wherein the mix contains between 70 and 80% by weight potassium nitrate, 10 to 25% activated charcoal and between 5 and 20% particulate sulphur.

7. A mouth wash, sealed sac or method as claimed in claim 1 wherein proportions of potassium nitrate, activated charcoal and particulate sulphur are 75:15:10.

8. A sac as claimed in claim 2 containing between 5 and 200 g of the mix.

9. A sac as claimed in claim 8 containing between 30 and 150 g of the mix.

10. A sac as claimed in claim 9 containing between 55 and 110 g of the mix.

11. A method as claimed in claim 4 wherein the sac is immersed in between 50 and 750 ml of water.

12. A method as claimed in claim 11 wherein the quantity of water is between 250 and 500 ml.

13. A method as claimed in claim 13 wherein the quantity of water is between 300 and 420 ml.

14. A method as claimed in claim 4 wherein the predetermined period of time is between 2 and 5 minutes.

15. A sac as claimed in claim 2 produced from a woven cloth or paper.
